(19) United States
(12) Patent Application Publication
(10) Pub. No.: US 2017/0326134 A1
(43) Pub. Date: Nov. 16, 2017

(54) Abrasion-Resistant Opioid Formulations

(71) Applicant: Relmada Therapeutics, Inc., New York, NY (US)

(72) Inventors: Fai JIM, Franklin Square, NY (US); Huai-Hung D. Kao, Syosset, NY (US)

(73) Assignee: Relmada Therapoutics, Inc., New York, NY (US)

(21) Appl. No.: 15/456,689

(22) Filed: Mar. 13, 2017

(30) Foreign Application Priority Data
May 11, 2016 (US) PCT/US16/31796

(51) Int. Cl. A61K 31/485; A61K 9/48; A61K 9/00

(52) U.S. Cl. CPC A61K 31/485; A61K 9/4875; A61K 9/4866; A61K 9/0053; A61K 9/485; A61K 9/4808; A61K 9/4858

(57) ABSTRACT

The disclosure relates to dosage forms which include one or more cohesion agents in amounts effective to reduce the likelihood and ease of extraction of an opioid agonist therefrom. The dosage forms exhibit improved resistance to abuse and lesser likelihood of accidental overdosing than similar dosage forms lacking a cohesion agent. Dosage forms including multiple cohesion agents capable of inhibiting or reducing extraction, abuse, or overdose over a broad range of temperatures are disclosed.

### CROSS-REFERENCES TO RELATED APPLICATIONS

This application claims priority under 35 U.S.C. 119 and the Paris Convention for the Protection of Intellectual Property to international application PCT/US16/31796, filed 11 May 2016.

### BACKGROUND OF THE DISCLOSURE

This disclosure relates generally to the field of abuse-resistant pharmaceutical compositions of opioid agonists, including orally administrable dosage forms.

The disclosure further relates to pharmaceutical compositions of opioids and their use for the treatment of pain, including compositions formulated for extended release of opioids (e.g., over a period of 8-48 hours). The technology disclosed herein can inhibit, reduce, prevent, or minimize the likelihood of opioid abuse or opioid toxicity from intentional tampering with or unintentional damage to opioid-containing dosage forms.

Medical practitioners attempting to alleviate and/or prevent pain can select from several well-accepted classes of pharmaceutical agents, including opioid analgesics. An important goal of analgesic therapy is to achieve continuous relief of pain. Regular administration of an analgesic is generally required to ensure that the next dose is given before the effects of the previous dose have worn off.

Conventional (so called “immediate-release,” “rapid release,” or “short acting”) opioid analgesics have been demonstrated to provide short-lived plasma levels, thereby requiring dosing every 4-6 hours in chronic pain. In contrast, extended release oral opioids are designed to maintain effective plasma levels throughout a 12 or 24-hour dosing interval. Extended release opioid formulations have been used by others in care for the management of chronic pain.

An important drawback with the use of opioids is the risk of drug addiction, drug diversion, and drug abuse. Furthermore, intentional tampering with or inadvertent damage to extended release formulations can result in rapid delivery of a massive dose and production of a variety of serious or life-threatening side effects, including respiratory depression and failure, sedation, cardiovascular collapse, coma, and death. Although the use of opioids for non-medical purposes has existed throughout recorded human history, their abuse has increased significantly in recent decades.

Addicts and recreational drug users can administer extended release opioids by a variety of routes. Commonly used methods include 1) parenteral (e.g., intravenous injection), 2) intranasal (e.g., snorting), and 3) episodic or repeated oral ingestion of intact or crushed tablets or capsules. Dosage forms including opioid analgesics may be ingested whole, crushed and ingested, crushed and vaporized or snorted, or injected intravenously after attempted extraction of the active pharmaceutical ingredient.

One mode of abuse involves the extraction of the opioid component from the dosage form by first mixing an opioid-containing table or capsule with a suitable solvent (e.g., water or alcohol) and then filtering or extracting the opioid from the mixture. Another mode of abuse of extended release opioids involves dissolving the drug in water, alcohol or another solvent to hasten its release and to ingest the solvent and drug orally. Extraction of opioid from the dosage form using a solvent depends on the kinetics of solid-to-liquid transfer, which are dependent in part upon the area of contact surface at the liquid/solid interface. For a given mass of drug formulation, particulate or powdered forms of the mass exhibit a far greater surface area than a rounded or flattened lump of the formulation. For this reason, individuals seeking to efficiently extract opioids from a drug formulation will often attempt to powder, finely abrade, or divide the formulation to yield such high-surface-area forms. High surface area compositions, like powders, can also be directly ingested, such as by swallowing a slurry or particles or by nasally inhaling a powder to deliver the powder to the nasal membranes or other portions of the respiratory system.

A number of strategies have been introduced to minimize the abuse of mood altering drugs such as opioids. Primary among these schemes is a legal infrastructure that controls the manufacture, distribution and sale of such drugs. Another strategy involves inclusion of an opioid antagonist in opioid-containing dosage forms intended for oral administration. The antagonist is not orally active, but substantially blocks the effects of the opioid if a user attempts to dissolve the opioid and administer it parenterally or nasally. Another version of this strategy involves inclusion in the oral dosage form of a sequestered, orally bioavailable opioid antagonist which is released only upon product tampering (e.g., crushing, extraction). In this circumstance, the opioid antagonist is not expected to be orally active under normal conditions of use but would nullify the euphoriant effects of either oral or intravenous administration upon product tampering. There is a need for a “passive” abuse deterrent system to protect both medical and non-medical users of opioids from intentional or unintentional opioid toxicity, without unnecessary harm to either group from the abuse deterrent technology.

Another abuse deterrent strategy involves including one or more aversive substances in pharmaceutical compositions containing opioids.

Formulations of extended release opioids may be vulnerable to dose dumping when co-ingested with alcohol, dose dumping being relatively rapid release (and corresponding rapid increase in blood levels) of opioids when co-ingested with alcohol, relative to their release in the absence of ethanol co-ingestion. There is a need, therefore, for methods of preventing the dose dumping effect of alcohol co-ingestion on opioid-containing compositions.

In summary, attempts have been made and are described in prior art to develop abuse-deterrent dosage forms. Clearly there is a need for a delivery system for commonly used oral dosage formulations of opioid drugs which deters intentional abuse, accidental alteration of opioid release kinetics from the dosage form, and preferably reduces the potential for psychological dependence upon opioids. In particular, there is a need for formulations that simultaneously provide robust abuse deterrence properties and an extended release pharmacokinetic profile suitable for oral administration of an opioid-containing dosage form every 12-24 hours. Among the favorable properties of such a dosage form are that the formulation i) provides a extended release pharmacokinetic profile suitable for every 12 or 24 hour release ii) resists crushing and abrasion, either at room temperature or upon freezing, iii) optionally, resists melting that might allow filtration of the formulation, its aspiration into a syringe, or extraction with a solvent, iv) if melted, inhibits extraction of opioids from the melted formulation, and v) optionally, avoids use of aversive agents or opioid antagonists.

The compositions and methods described herein exhibit these favorable characteristics.

### BRIEF SUMMARY OF THE DISCLOSURE

The disclosure relates to a pharmaceutical dosage form for orally administering an opioid agonist to a human. The dosage form includes a matrix. The matrix includes a therapeutically effective amount of the opioid agonist, one or more abuse deterrent, extended release (ADER) ingredients, and one or more cohesion agents. The ADER ingredient(s) can, for example, be hydrogenated vegetable oils, polyoxyethylene stearates, polyoxyethylene distearates, glycerol monostearate, and poorly water soluble, high melting point waxes. The cohesion agent(s) should be present in an amount sufficient, at at least one temperature in the range −20 to 100 degrees Celsius, to increase either (or both) of the stickiness and the elasticity of the matrix by at least about 5%, relative to the same matrix lacking the cohesion agent. The cohesion agent(s) can confer a sticky consistency or an elastic consistency to the matrix (or both). These matrix components can be present as a substantially homogenous mixture, for example.

Cohesion agent useful in these compositions include, for example, natural rubbers, synthetic rubbers, silicones polymers, vegetable gums, paraffins, lanolins, mineral oils, gelling agents, and mucilages.

Substantially any opioid agonist can be included in the matrix and will be less susceptible to intentional or accidental abuse, misuse, and extraction that the agonist would be in a similar matrix lacking the cohesion agent(s). For example, the opioid agonist can be one or more of buprenorphine, butorphanol, levorphanol, methadone, and tramadol.

### DETAILED DESCRIPTION

The disclosure relates to pharmaceutical dosage forms which are formulated to release an opioid at a rate that provides a therapeutic quantity to a human subject over an extended period of time (e.g., for more than four hours, preferably for about 12-24 hours) following oral administration of the dosage form and which also exhibit abuse deterrence properties which inhibit release of the opioid from the dosage form at a more rapid rate, whether that more-rapid release is occasioned by intentional manipulation of the dosage form or by unintentional damage to or co-ingestion of the dosage form with another agent, such as ethanol. By way of example, the disclosure relates to dosage forms intended for oral administration and suitable for multiple-times-per-day up to once-a-day (e.g., Q4H, Q6H, Q8H, Q12H, and Q24H) administration.

The dosage forms described herein include one or more opioids dispersed within a matrix. The matrix includes at least an extended release material selected such that, upon contacting a selected fluid in the GI tract, a therapeutically effective amount of the opioid(s) is released for a period of at least 4 hours, and not longer than 48 hours. Preferably, the opioid(s) is released in a therapeutically effective amount for from about 6-24 hours, 8-24 hours, or more preferably for about 12-24 hours. The matrix also includes at least one cohesion ingredient in an amount effective to inhibit the effectiveness of common methods of extracting opioid(s) from pharmaceutical dosage forms, such as crushing, grinding, and extracting with a solvent.

The matrix can include one or more ingredients that confers upon the dosage form the property that release of the opioid(s) from the dosage form extends over an extended period of time, such as from 4-48 hours. The matrix can also include one or more ingredients that confers upon the dosage form the property that deliberate or unintentional damage to the dosage form does not drastically (or, in some embodiments, even significantly) increase the rate at which the opioid(s) is released from dosage form, thereby rendering the dosage form relatively resistant to abuse. In another embodiment, the matrix includes one or more ingredients that substantially prevents release of the opioid(s) from the dosage form for at least about 15 or 30 minutes. Such ingredients are referred to herein as ADER (abuse deterrent, extended release) ingredients. Examples of suitable ADER ingredients include (a) hydrogenated vegetable oils; (b) polyoxyethylene stearates and distearates; (c) glycerol monostearate; (d) poorly water soluble, high melting point waxes (i.e., those having melting points from about 40 to 100 degrees Celsius). Dosage forms (and opioid-containing formulations within such dosage forms) can include a single ADER ingredient or mixtures of ADER ingredients. ADER ingredients are further described in U.S. Patent Application Publication number 2009/0082466.

The dosage forms described herein include a cohesion agent (or multiple cohesion agents) which imparts one or more of the following properties to the opioid-containing formulation of the dosage form at a routinely-attainable temperatures (e.g., from −20 to 100 degrees Celsius): i) the agent increases the resistance of the opioid-containing formulation of the dosage form to powdering when the formulation is crushed; ii) the agent increases the resistance of the opioid-containing formulation of the dosage form to breakage or division when the formulation is subjected to cutting using, for example, a knife or razor blade; iii) the agent increases the cohesion (in the materials-science sense) of the opioid-containing formulation of the dosage form; and iv) the agent increases the stickiness of the opioid-containing formulation of the dosage form (i.e., adhesion, in the chemical sense, between the formulation and common materials, such as steel of a knife or razor blade, or between particles of the formulation itself). Preferably the cohesion agent(s) imparts more than one of properties i-iv to the formulation (relative to the same formulation lacking the cohesion agent).

Some ADER ingredients are also able to act as cohesion agents (e.g., some hydrogenated vegetable oils will also cause a formulation containing them to resist powdering). However, not all cohesion agents will necessarily affect the rate of release of opioid(s) from the formulations described herein. Thus, while all cohesion agents will necessarily confer abuse resistance of at least one of the types described herein to opioid-containing formulations, not all cohesion agents are ADER ingredients.

An important goal of the compositions described herein, in addition to providing release of therapeutic amounts of an opioid from a dosage form when administered as intended (e.g., orally), is to reduce the likelihood and/or degree of opioid release from the compositions that may be generated as a result of intentional or unintentional physical damage to the dosage form or as a result of interaction of chemicals (e.g., ethanol or other solvents) with the dosage form. By way of example, the dosage form is intended to resist deliberate attempts to extract opioid therefrom, such as by crushing, breaking, shearing, abrading, grinding, milling, powdering, chewing, dissolving, melting, mechanically extracting, or chemically extracting the dosage form. Further by way of example, the dosage form is intended to resist altered opioid release attributable to unintentional damage to the dosage form, such as by shipping-related breakage, incidental or accidental dental abrasion of the dosage form during oral administration, and unintended interaction between the dosage form and co-ingested chemicals or solvents (e.g., ethanol).

The dosage forms described herein can be used to treat or prevent diseases and disorders amenable to treatment with opioid agonists, including pain. Inclusion of an ADER ingredient extends the period of time over which a therapeutically effective amount of the opioid is administered to a patient who consumes the dosage form. Inclusion of one or more cohesion agents reduces the likelihood that the opioid in the dosage form can or will be used in non-intended ways, such as through non-medical, recreational use or by maladministration attributable to inadvertent dosage form damage.

Release of opioid from the dosage forms described herein is preferably controlled primarily by the rate at which the opioid is released within the gastrointestinal (GI) tract upon swallowing of the dosage form in its whole, uncompromised state. The dosage form can, for example, be coated with an enteric coating so that little or none of the opioid will be released in the stomach, the opioid instead being released in portions of the GI tract more distal to the mouth. Alternatively, or in addition, dissolution of the coating (and, consequently, initial release of opioid) can be made pH-dependent, so that such dissolution occurs primarily or only in regions of the GI tract having a selected pH, and/or time-dependent, so that such dissolution occurs by a selected time following oral administration of the dosage form.

The dosage form can, for example, include a single unitary matrix (e.g., an oblong or spherical capsule-shaped, opioid-containing matrix, whether contained within a capsule, coated, or uncoated) from which the opioid diffuses, either through the matrix (or pores within the matrix) or as the matrix itself dissolves in the GI tract. Alternatively, the dosage form can include a capsule shell which readily dissolves within the GI tract, the capsule shell including multiple particles of an opioid-containing matrix (each of the particles comprising the same matrix or different matrices), so that the opioid release is a two-step process, the first step involving release of the particles from the capsule shell and the second step involving release of the opioid from the particles. Capsule-within-a-capsule configurations can also be used. Combinations of these alternatives can be employed as well.

In one embodiment, the dosage form includes multiple particles of an opioid-containing matrix (each of the particles comprising the same matrix or different matrices) suspended in a digestible mass. The opioid-containing particles also include a cohesive material in the matrix in an amount sufficient to confer a sticky texture to the matrix particles when they are released from the mass by digestion. If multiple particles are released from the mass in a confined space (e.g., in the stomach or in a glass or beaker containing vinegar or simulated gastric fluid), the released particles will tend to stick to one another, coalescing in a conglomerate that will exhibit a significantly lower surface area than the combined surface areas of the individual particles, thereby decreasing the rate of release of the opioid from the particles. Such a dosage form can be useful for preventing abuse, such as accidental or intentional ingestion of multiple dosage forms or attempts to extract the opioid from the dosage form outside the body.

In other embodiments, the dosage form of the invention comprises a compressed tablet, compressed capsule or uncompressed capsule. In other embodiments, the dosage form comprises a liquid fill capsule. In a preferred manufacturing method, the opioid-containing formulation is solid (even if a flowable, viscous solid having viscosity greater than about 50,000 or 100,000 Centipoise) at the normal human body temperature of 37 degrees Celsius, but is flowable (has a viscosity not greater than about 150,000 Centipoise) at a higher temperature (e.g., at 40 degrees Celsius, or at any temperature in the range 40-100 degrees Celsius). Such dosage forms can be made by filling an empty capsule shell with the flowable formulation at a temperature greater than 40 degrees Celsius and then cooling it to room temperature of about 20 degrees Celsius, for example.

In some preferred embodiments, the dosage form of the invention comprises an oral formulation (e.g., tablet or capsule) which is coated to prevent substantial direct contact of opioid with oral cavity (e.g. tongue, oral mucosa), oropharyngeal mucosal surface, esophagus or stomach. In some preferred embodiments, the dosage form is an oral formulation which is coated with a film or polymer. The dosage form of the invention can include one or more opioids contained within an enteric coating. The dosage form can include one or more opioids formulated with pharmaceutical excipients and auxiliary agents known in the art, such that the opioid is released after an approximate selected amount of time, or at an approximately specific anatomic location in the gastrointestinal tract (e.g., within one or more of the stomach, the ileum, the jejunum, the duodenum, and the colon), or when the dosage form is in contact with specific gastrointestinal conditions (e.g., pH range, osmolality, electrolyte content, food content).

The disclosure is also directed to method of treating or preventing diseases and disorders amenable to treatment with opioid agonists, including pain with the dosage forms disclosed herein. Opioid agonists are known to be effective for treatment, inhibition, and prevention of various types of pain, including, for example, central and peripheral neuropathic pain, back pain, chronic pain, pain associated with osteoarthritis, cancer, or fibromyalgia, and chronic inflammatory pain.

The methods include providing the oral dosage form containing an opioid agonist, with the agonist being available for immediate release following administration, for extended release, or for both immediate and extended release. That is, the dosage form can include both a first aliquot of the agonist that is formulated for substantially immediate release upon reaching a desired GI tract location (e.g., the stomach or the colon) and a second aliquot of the agonist that is formulated for extended release following the immediate release. The two aliquots can include the same or different opioid agonists.

Further details of the dosage forms are described separately in sections below.

The Opioid Agonist

The dosage form includes one or more opioids agonists. Each opioid agonist can be included in an unsalified form (e.g., as an opioid base) or in the form of a pharmaceutically acceptable salt, ester, solvate, complex, hydrate, or other conventionally-available form. Furthermore, opioid agonists can be included in racemic form or as an individual diastereoisomer or enantiomeric isomer thereof. Nonlimiting examples of conventional pharmaceutical salts of opioid agonists include hydrochlorides, hydrobromides, hydroiodides, sulfates, bisulfates, nitrates, citrates, tartrates, bitartrates, phosphates, malates, maleates, napsylates, fumarates, succinates, acetates, terephthalates, pamoates and pectinates.

The amount of opioid agonist included in the oral dosage form is not critical, and calculation of therapeutic amounts is within the ken of a skilled artisan in this field, taking into account the therapy being performed, the duration of therapeutic effect desired, and the expected release rate of the agonist from the dosage form when orally administered. The amount will vary depending on variety of physiologic, pharmacologic, pharmacokinetic, pharmaceutical and physicochemical factors, including: (i) whether the opioid is supplied as the base, as pharmaceutically acceptable salt or another form, or as a mixture of these; (ii) the nature of the oral dosage form (e.g., whether immediate release and/or extended release aliquots are included); (iii) the anatomical location of the pain relieving target; (iv) the intensity and intractability of the pain; (v) the contribution of different mechanism to the initiation, propagation, summation and maintenance of the pain; (vi) the absorption, metabolism, distribution and excretion of orally administered opioids in healthy subjects and in patients with various diseases and disorders, including renal and hepatic impairment; (vii) the presence of comorbid pathology; (viii) the patient's risk of iatrogenic side effects; (ix) the tolerability of the dose, including the patient's propensity for opioids associated side effects; (x) use of concurrent analgesics; (xi) the efficiency of the dosage form; and (xii) the physicochemical properties of the opioid, including its solubility and hydrophilicity. Suitable amounts of opioid agonists can, for example, be in the range from about 10 picograms to 1.500 grams. More common ranges include about 0.1 microgram to 1000 milligrams, about 0.1 microgram to 500 milligrams, about 0.1 microgram to 250 milligrams, or about 1 microgram to 100 milligrams.

Therapeutic effectiveness of an opioid agonist, as used herein, means satisfactory prevention, reduction in, or elimination of neuropathy or pain, together with a tolerable level of side effects, as determined by the human patient.

Substantially any opioid agonist can be included in the dosage forms described herein. Examples of known, suitable opioid agonists include alfentanil, allylprodine, alphaprodine, anileridine, apomorphine, apocodeine, benzylmorphine, bezitramide, brifentanil, buprenorphine, butorphanol, carfentanil, clonitazene, codeine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, mirfentanil, morphine, myrophine, nalbuphine, narceine, nicomorphine, norlevorphanol, normethadone, nalorphine, nociceptin/orphanin FQ (N/OFQ), normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanil, sufentanil, tapentadol, tramadol, trefentanil, and tilidine. Preferred opioid agonists include buprenorphine, butorphanol, levorphanol, methadone, and tramadol.

The opioid can be included in an immediate release form, in addition to an extended release form. When the opioid is included in an immediate release form, it can, for example, be coated onto a substrate of the dosage form. For example, where the extended release of opioid from the dosage is attributable to a controlled release coating, the immediate release layer can be over-coated atop the controlled release coating. Further by way of example, in a dosage form in which a plurality of sustained release substrates which include the opioid are incorporated into a hard gelatin capsule, the immediate release portion of the opioid can be incorporated into the gelatin capsule as a powder, liquid, or granulate within the capsule or as a coating on the exterior or interior of the capsule shell.

ADER Ingredients

The dosage form includes a therapeutically effective amount of one or more opioid agonists and one or more abuse deterrent, extended release (ADER) ingredients, which are selected from among (a) hydrogenated vegetable oils; (b) polyoxyethylene stearates and distearates; (c) glycerol monostearate; and (d) poorly water soluble waxes which exhibit high melting point (40-100 degrees Celsius).

Suitable hydrogenated vegetable oils of the present invention may include hydrogenated cottonseed oil (e.g., Akofine®; Lubritab®; Sterotex® NP), hydrogenated palm oil (e.g., Dynasan® P60; Softisan® 154), hydrogenated soybean oil (e.g., Hydrocote®; Lipovol HS-K®; Sterotex® HM) and hydrogenated palm kernel oil (e.g., Hydrokote® 112).

Suitable polyoxyethylene stearates and distearates of the present invention include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag® 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g., Lipo-PEG® 100-S; Myrj® 59; Hodag® 600-S; Ritox® 59; Hodag® 22-S; PEG4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one embodiment, the opioid is combined with beeswax, hydroxypropyl methyl cellulose (e.g., HPMC K15M), silicon dioxide (alone or in combination with Al₂O₃; e.g., Aerosil®, Aerosil® 200, Aerosil® COK84). Alternatively, the opioid can be combined with hydrogenated cottonseed oil (e.g., Sterotex® NF), hydroxypropyl methyl cellulose (e.g., HPMC K15M), coconut oil, and silicon dioxide (alone or in combination with Al₂O₃; e.g., Aerosil®, Aerosil® 200, Aerosil® COK84). In embodiment, the opioid is combined with glycerol monostearate (e.g., Cithrol® GMS), hydroxypropyl methyl cellulose (e.g., HPMC K100M) and silicon dioxide (alone or in combination with Al₂O₃; e.g., Aerosil®, Aerosil® 200, Aerosil® COK84). In still another preferred embodiment, the opioid is combined with hydrogenated palm kernel oil (e.g., Hydrokote® 112), hydroxypropyl methyl cellulose (e.g., HPMC K15M) and silicon dioxide (alone or in combination with Al₂O₃; e.g., Aerosil®, Aerosil® 200, Aerosil® COK84).

One or more release rate modifiers can be included in the dosage form, including hydroxypropyl methyl cellulose (e.g., HPMC K15M) may be incorporated. Release rate modifiers can alter the rate at which the opioid(s) are released from the dosage form and can also have additional useful properties, such as imparting viscosity or tack when the dosage form is combined with liquid or increasing the viscosity or tack of the dosage form when it it melted.

Thixotropes (e.g., fumed silicon dioxides, Aerosil®, Aerosil® COK84, Aerosil® 200, etc.) can be incorporated into the dosage form. Thixotropes enhance the pharmaceutical formulations of the invention by increasing the viscosity of solutions during attempted extraction, complementing the action of HPMCs.

The dosage form can include one or more ADER agents. Any amount of ADER ingredients can be used, but the amount is preferably selected both to yield favorable abuse-deterring and opioid-release-extending properties, in addition to yielding practically administrable dosage forms (e.g., capsules small enough to be swallowed by ordinary humans). In some embodiments, the total amount of ADER ingredients in the dosage form is about 5 to about 98 percent, preferably 7 to 90 percent, and more preferably 10 to 85 percent on a dry weight basis of the dosage form.

Upon contact with a solvent (e.g., water), ADER agents can absorb the solvent and swell, thereby forming a viscous or semi-viscous substance that significantly reduces and/or minimizes the amount of free solvent which can contain an amount of solubilized drug. This can also reduce the overall amount of drug extractable with solvent by entrapping the drug in a matrix.

The rate of opioid release from the dosage forms described herein can assessed using by the USP Basket and Paddle Method (USP-28 NF-23, 2005, as published by the United States Pharmacopeial Convention, Inc.) at 100 rotations per minute in 700 milliliters of Simulated Saliva (per USP, without enzymes), Simulated Gastric Fluid (SGF, per USP), or Simulated Intestinal Fluid (SIF, without enzymes, per USP) at 37 degrees Celsius, and measuring release of opioid from the dosage form at selected times thereafter (e.g., after one hour of treatment by this method). For orally-administered opioids, it can be desirable that little or none of the opioid is released within the oral cavity during administration. For certain opioids, it is also preferable that little or none of the opioid is released within the stomach, or that most of the opioid is released within certain portions of the small or large intestines. These conditions can be simulated using the USP Basket and Paddle Method using fluids appropriate to model the desired GI tract compartments (a skilled artisan would understand and can select such fluids) and residence times (e.g., a skilled artisan understands that appropriate residence times in various GI tract compartments can depend on the feeding state of an individual, and thus upon whether the dosage form is intended to be taken with food).

The dosage forms described herein can be made to release the opioid(s) contained therein over an extended period of time. Design of such dosage forms is understood to be, in part, empirical, taking into account the ADER ingredients selected for the dosage form, the opioid(s) to be released, the other ingredients of the dosage form (including the cohesion agent described herein), and the period of time over which opioid release is to be effected. Based on this information, a skilled artisan can develop an approximate dosage form composition that is expected to be effective, test the composition (e.g., using the USP Basket and Paddle Method described herein with appropriate testing fluids to model the expected or desired site of release), and refine the approximate composition to more nearly deliver the desired release profile. This process can be repeated iteratively several times to yield a refined composition that includes the desired ingredients and exhibits the desired opioid release profile. By way of example, the process can be used to make a dosage form which includes an opioid agonist, one or more ADER ingredients, and a cohesion agent and which releases, by way of examples: a) a therapeutic amount of the opioid beginning substantially immediately after oral administration and continuing for about 4, 8, 12, 16, 20, 24, 36, or 48 hours thereafter; b) a therapeutic amount of the opioid beginning not sooner than about 1 hour after oral administration and continuing for about 8, 12, 16, 20, 24, 36, or 48 hours thereafter; or c) a therapeutic amount of the opioid beginning substantially immediately after oral administration and, beginning about two hours thereafter, further therapeutic amounts of the opioid continuing for about 4, 8, 12, 16, 20, 24, 36, or 48 hours thereafter.

Cohesion Agents

The dosage form includes at least one cohesion agent in an amount sufficient to inhibit or reduce intentional division of the opioid-containing portion of the dosage form into high-surface area compositions, such as powders or thin films. By inhibiting or reducing increase of the surface area of that portion, the cohesion agent stabilizes the release rate of the opioid from the dosage form and inhibits or reduces intentional extraction or abuse of the opioid.

The cohesion agent inhibits or reduces crushing, division, spreading, stretching, or disaggregation of the matrix (i.e., makes it more difficult and/or time-consuming, or less possible, to perform any of these manipulations), by enhancing binding and/or bonding of the opioid-containing matrix to itself. This has the effect of inhibiting or reducing the ability of an individual to increase the surface area of the opioid-containing portion, such as for the purpose of extracting the opioid therefrom. This also has the effect of reducing the amount of powder that is produced when the matrix is crushed, abraded, ground, chopped or sliced with a blade and of generally increasing the particle size of any such powder that can be produced (finer powders generally have greater surface area per unit mass than coarser powders). This can furthermore have the effect of causing thin sheets or strands of the matrix that are transiently generated during pressing, grinding, or stretching of the matrix to retract into coarser, lower-surface-area particles or lumps.

Use of cohesion agents to inhibit or reduce powdering and disintegration is known in a general sense. Indeed, binding agents are frequently used to enable formation of tablets from powders upon compression of a powder including both a drug and a binding agent such as starch. However, it was not previously recognized that one or more cohesion agents ought to be incorporated into abusable drug dosage forms in order to inhibit or reduce abuse—whether, for example, by direct administration of a powdered dosage form or extraction of the drug from the dosage form followed by subsequent administration of the extracted drug. Moreover, use of a plurality of cohesion agents to inhibit or reduce abuse and/or extraction of drugs over a range of readily-available temperatures (e.g., about −20 to 100 degrees Celsius) has not been previously described.

Described herein are cohesion agents which can inhibit or reduce extraction of an abusable drug from a dosage form of that drug (e.g., a commercially available dosage form modified to include the cohesion agent(s)). The types of extraction that can be inhibited or reduced include one or more of increasing the surface area of the drug-containing portion of the dosage form, contacting a solvent with the surface of the portion to thereby extract the drug for abusive use, melting the drug-containing portion, and dissolving the drug-containing portion in a solvent. The cohesion agents described herein can also inhibit or reduce abuse from a dosage form of a drug that is effected by increasing the surface area of the drug-containing portion of the dosage form and administering that increased-surface-area-portion to an abuser. In each of these instances, the cohesion agents described herein make it more difficult to increase the surface area of the dosage form, the solubility of the drug in a solvent, or both, whether for drug-extraction or direct drug-abuse.

Generally speaking, one type of the cohesion agents described herein tend to be compounds or mixtures which increase the stickiness or pastiness of a drug-containing portion of a dosage form. In some embodiments, the cohesion agent(s) increase the stickiness or pastiness of the portion relative to the same portion lacking the cohesion agent(s). In other embodiments, the cohesion agent(s) increase the stickiness or pastiness of the portion when that portion is combined with a solvent (e.g., water, ethanol, or vinegar), relative either to the stickiness of the portion lacking the cohesion agent(s) or to the stickiness of the portion containing the cohesion agent when the solvent is not present. By increasing the stickiness or pastiness of the abusable-drug-containing portion of a dosage form, the cohesion agent(s) decrease the likelihood that one seeking to extract or abuse the drug from the dosage form will be able to enhance the rate or extent of extraction or drug release from the portion, such as by crushing, division, or solvent-extraction of the portion.

Cohesion agents which increase the stickiness or pastiness of an abusable drug-containing composition tend to be agents that are waxy, gum-like, or highly viscous liquids (i.e., liquids having a viscosity of about 200 to 250,000 centipoise (cP), more preferably about 500 to 150,000 cP, and even more preferably about 2,000 to 100,000 cP) at at least one temperature in the range −20 to 100 degrees Celsius, such as at room temperature (ca. 20 degrees Celsius). When combined with an abusable drug and one or more ADER ingredients at a temperature in this range, cohesion agents of this type can render the composition a sticky, coherent mass that is more difficult to diaggregate into small particles, difficult to spread into a thin layer, or both.

Examples of materials which can be combined with an abusable drug and one or more ADER ingredients to yield compositions with these consistencies include paraffins, lanolins, mineral oils, vegetable gums, viscosity enhancers (e.g., polyacrylic acids such as those marketed under the Carbomer® trademark, chitosans, polyvinyl alcohols, and polyethylene oxides), long chain glycerides (preferably those having a melting point lower than 40 degrees Celsius), gelling agents (e.g., chitosans, glyceryl monooleate, glyceryl palmitostearate, locust bean gum, and gelatin), and mucilages (e.g., natural and synthetic mucilages, methylcellulose, and carboxymethylcellulose).

Stickiness (i.e., tackiness or tack) of an opioid-containing matrix can be assessed by substantially any known method. By way of example, testing method ASTM D2979-01(2009), Standard Test Method for Pressure-Sensitive Tack of Adhesives Using an Inverted Probe Machine (ASTM International, West Conshohocken, Pa.; herein “the inverted probe method”) can be used to assess the stickiness of a selected amount (e.g., 100 milligrams) of the matrix that contains a selected quantity of the cohesion agent and compared with the stickiness (assessed using the same method and equipment) of the matrix lacking the cohesion agent. Such testing should be performed using flat stainless steel contact surfaces and assessed at a controlled temperature (i.e., −20 to 100 degrees Celsius) after compressing the matrix between the contact surfaces under 25 pounds of pressure for ten seconds, for example. An enhancement of at least 1% (preferably at least 2%, 3%, 5%, 10%, 20%, 50%, 100%, or 200%) in the amount of force required to subsequently separate the contact surfaces is desirable.

Another type of cohesion agents that can be used are materials which confer a resiliently-retracting (elastic) or rubbery consistency (e.g., like chewing gum or the eraser of a common pencil) to a composition that includes an abusable drug, one or more ADER ingredients, and the cohesion agent. Examples of materials of this type include elastomers (e.g., natural and synthetic rubbers and silicone polymers), vegetable gums (e.g. acacia, agar, guar, and xanthan gums, gum Arabic, tragacanth, and other known gum bases), hydrophilic polymers (e.g., starches, carrageenan, chitosans, latexes, and polypeptides such as zeins, collagens, gelatins, and glutens), beeswax, and dibutyl sebacate).

Elasticity (i.e., resilient retraction after stretching) of an opioid-containing matrix can be assessed by substantially any known method. By way of example, the following testing method can be used to assess the elasticity of the matrix. A selected amount (e.g., one gram) of the matrix that contains a selected quantity of the cohesion agent is formed into a defined shape (e.g., a cylinder having a diameter of 5 millimeters), fixing the cylinder into a pair of spaced grips, moving the grips a defined distance (e.g., increasing by 5% of the distance between the grips), and assessing the tension force exerted on the grips following such movement. This measurement can be compared with the elastic tension (assessed using the same method and equipment) of the matrix lacking the cohesion agent. Such testing should be performed at a controlled temperature (i.e., −20 to 100 degrees Celsius). An enhancement of at least 1% (preferably at least 10%, 50%, or 200%) in the amount of elastic tension force is desirable.

Alternatively, elasticity can be measured using a standard texture analyzer device in order to determine the breaking point of an opioid-containing matrix (herein, “the texture analyzer method”). By way of example, such a matrix is placed on the platform of a Stable Microsystems Texture Analyzer TA-XT Plus device (marketed by Stable Micro Systems Ltd., Surrey, UK), and a force at a controlled temperature (i.e., −20 to 100 degrees Celsius) at a specific speed is applied to the matrix. Matrices including one or more cohesion agents will exhibit greater elasticity than matrices lacking the cohesion agent(s) and will be more resistant to breakage by compression. Resistance to breakage can be measured either in terms of a greater distance needing to be traveled to reach the breaking point or more force needing to be applied to reach the breaking point. An enhancement of at least 1% (preferably at least 2%, 3%, 5%, 10%, 20%, 50%, 100%, or 200%)) in either the distance traveled and/or the force applied is considered suitable.

A dosage form as described herein can include multiple cohesion agents. In one embodiment, the dosage form includes one or more cohesion agents which increase the stickiness or pastiness of a drug-containing portion of a dosage form and also includes one or more cohesion agents which confer a resiliently-retracting or rubbery consistency. Preferably, at least one of these consistencies is exhibited at every temperature in the range from −20 to 100 degrees Celsius (which represents temperatures easily achieved by recreational abusers who may seek to extract opioid from the dosage form). By way of example, an opioid-containing dosage form can include a substantially homogenous matrix that includes an opioid agonist, an ADER ingredient, and two cohesion agents, including both a paraffin material that is a relatively stiff, waxy substance at temperatures below about 20 degrees Celsius, but a sticky, viscous fluid at temperatures greater than about 20 degrees Celsius and a vegetable gum that is a resilient, rubbery material at temperatures from about −20 to 30 degrees Celsius, but which melts to form a viscous fluid at temperatures greater than about 30 degrees Celsius. When such a dosage form is maintained at temperatures from about −20 to 20 degrees Celsius, the paraffin cohesion agent is a waxy solid and would normally be disaggregatable into small particles by abrasion or chopping into fine particles using a blade; however, the vegetable gum cohesion agent exhibits a rubbery consistency at these temperatures, inhibiting or reducing abrasion or fine chopping of the matrix. When the same dosage form is maintained at a temperature between about 20 and 30 degrees Celsius, both the viscous nature of the paraffin cohesion agent and the rubbery consistency of the vegetable gum cohesion agent inhibit or reduce disaggregation of the matrix. At temperatures of about 30-100 degrees Celsius, the disaggregation-inhibiting or—reducing efficacy of the vegetable gum cohesion agent decreases (owing to cessation of its elasticity), but the viscous, sticky nature of the paraffin cohesion agent nonetheless inhibits or reduces disaggregation of the matrix at these temperatures.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

### Example 1

Hydrogenated palm kernel oil is heated to a temperature of about 60 degrees Celsius. Glyceryl monooleate is added. Once a homogenous mixture is obtained, the remaining ingredients are added and mixed with a homgenizer to form a molten, flowable mixture, and the mixture is injected into an empty dosage form (e.g., a size 2 capsule shell). The mixture hardens as it cools, typically upon injection into the capsule shell.

| | Content | Quantity per Capsule |
|---|---|---|
| Ingredient | (% w/w) | in milligrams |
| Hydrogenated Palm Kernel Oil | 72.3 | 235 |
| HPMC | 18.5 | 60 |
| Colloidal Silicon Dioxide | 3.1 | 10 |
| Glyceryl monooleate | 3.1 | 10 |
| Levorphanol | 3.1 | 10 |
| Total Capsule Fill | | 325 |

In this formulation and those described in the other examples, Hydrokote® 112 can be used as the hydrogenated palm kernel oil (which is an ADER ingredient); “HPMC” is hydroxyproplymethylcellulose (such as the Methocel K15M product); the colloidal silicon dioxide can be a product such as Aerosil® 200; glyceryl monooleate (a cohesion agent) can be the Capmul® GMO product; and levorphanol is an opioid agonist.

### Example 2

Hydrogenated palm kernel oil is heated to a temperature of about 60 degrees Celsius. The remaining ingredients are added with mixing, while maintaining the temperature at about 60 degrees Celsius, to form a molten, flowable mixture. The mixture is injected into an empty dosage form (e.g., a size 1 capsule shell). The mixture hardens as it cools, typically upon infection into the capsule shell.

| | Content | Quantity per Capsule |
|---|---|---|
| Ingredient | (% w/w) | in milligrams |
| Hydrogenated Palm Kernel Oil | 50.8 | 235 |
| HPMC | 18.5 | 60 |
| Colloidal Silicon Dioxide | 3.1 | 10 |
| Dibutyl sebacate | 6.2 | 20 |
| Xanthan gum | 3.1 | 10 |
| Guar gum | 15.4 | 50 |
| Levorphanol | 3.1 | 10 |
| Total Capsule Fill | | 395 |

In this formulation, each of dibutyl sebacate, xanthan gum, and guar gum is a cohesion agent. In this formulation and those in the other examples, the dibutyl sebacate can be the Morflex® DBS product; the xanthan gum can be the Vanzan® product; and the guar gum can be the Edicol® 60-70 product.

### Example 3

Dilute lactic acid in 75 milliliters of water to form a 10% (v/v) acid concentration and add in sufficient chitosan to yield a 2% w/v chitosan/lactic acid solution. Separately heat yellow beeswax to about 70 degrees Celsius. Add the chitosan-citric acid solution to the molten yellow beeswax, followed by the remaining ingredients, and mix with a homogenizer to form a molten, flowable mixture. Inject the mixture into an empty dosage form (e.g., a size 2 capsule shell).

| | Content | Quantity per Capsule |
|---|---|---|
| Ingredient | (% w/w) | in milligrams |
| Yellow Beeswax | 49.3 | 165 |
| HPMC | 14.9 | 50 |
| Aerosil (RTM) | 3.0 | 10 |
| Chitosan | 1.5 | 5 |
| Lactic Acid | 7.5 | 25 |
| Gelatin | 20.9 | 70 |
| Water | — * | — * |
| Levorphanol | 3.0 | 10 |
| Total Capsule Fill | | 335 |

* Water is removed during the manufacturing process.

In this formulation, beeswax is an ADER ingredient, and each of chitosan and gelatin is a cohesion agent. Dissolution of chitosan in the acid solution triggers its viscous properties. In this formulation and those in the other examples, the chitosan can be the Chitopharm® M product, and the gelatin can be a type B gelatin exhibiting a bloom strength of 220.

### Example 4

Hydrogenated vegetable oil and fractionated coconut oil are heated to a temperature of about 60 degrees Celsius. The remaining ingredients are added with mixing, while maintaining the temperature at about 60 degrees Celsius, to form a molten, flowable mixture. The mixture is injected into an empty dosage form (e.g., a size 2 capsule shell).

| | Content | Quantity per Capsule |
|---|---|---|
| Ingredient | (% w/w) | in milligrams |
| Hydrogenated Vegetable Oil | 41.1 | 150 |
| Fractionated Coconut Oil | 20.5 | 75 |
| HPMC | 19.2 | 70 |
| Colloidal Silicon Dioxide | 2.7 | 10 |
| Acacia gum | 8.2 | 30 |
| Polyvinyl alcohol | 5.5 | 20 |
| Levorphanol | 2.7 | 10 |
| Total Capsule Fill | | 365 |

In this formulation, the hydrogenated vegetable oil (an ADER ingredient) can be a hydrogenated cottonseed oil such as Sterotex®; fractionated coconut oil is also an ADER ingredient and can be a product such as Miglyol® 812, and each of acacia gum (e.g., AgriSpray® Acacia R) and polyvinyl alcohol (e.g., Emprove® 40-88) is a cohesion agent.

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

What is claimed is:

1. A pharmaceutical dosage form for orally administering an opioid agonist to a human, the dosage form including a matrix comprising:
a therapeutically effective amount of the opioid agonist;
at least one abuse deterrent, extended release (ADER) ingredient selected from the group consisting of hydrogenated vegetable oils, polyoxyethylene stearates, polyoxyethylene distearates, glycerol monostearate, and poorly water soluble, high melting point waxes; and
at least one cohesion agent in an amount sufficient, at at least one temperature in the range −20 to 100 degrees Celsius, to achieve at least one of
i) increasing the stickiness of the matrix by at least about 5%, relative to the same matrix lacking the cohesion agent, as assessed by the inverted probe method and
ii) increasing the elasticity of the matrix as assessed by increasing the breaking force of the matrix, by at least about 5% by the texture analyzer method, relative to the same matrix lacking the cohesion agent.

2. The dosage form of claim 1, wherein the matrix comprises a substantially homogenous mixture of the opioid agonist, the ADER ingredient, and the cohesion agent.

3. The dosage form of claim 1, wherein the matrix comprises a single cohesion agent in an amount sufficient to achieve both i and ii.

4. The dosage form of claim 1, wherein the matrix comprises multiple cohesion agents in amounts sufficient to achieve both i and ii.

5. The dosage form of claim 1, wherein the matrix comprises a cohesion agent selected from the group consisting of natural rubbers, synthetic rubbers, silicones polymers, vegetable gums, paraffins, lanolins, mineral oils, gelling agents, and mucilages.

6. The dosage form of claim 1, wherein the matrix comprises a cohesion agent that confers an elastic consistency to the matrix.

7. The dosage form of claim 1, wherein the matrix comprises a cohesion agent that confers a sticky consistency to the matrix.

8. The dosage form of claim 7, wherein the matrix further comprises a cohesion agent that confers an elastic consistency to the matrix.

9. The dosage form of claim 1, wherein the matrix comprises a cohesion agent that confers both an elastic consistency and a sticky consistency to the matrix.

10. The dosage form of claim 1, wherein the opioid agonist is selected from the group consisting of buprenorphine, butorphanol, levorphanol, methadone, and tramadol.

11. A pharmaceutical dosage form for orally administering an opioid agonist to a human, the dosage form including a matrix comprising:
a therapeutically effective amount of the opioid agonist;
at least one abuse deterrent, extended release (ADER) ingredient selected from the group consisting of hydrogenated vegetable oils, polyoxyethylene stearates, polyoxyethylene distearates, glycerol monostearate, and poorly water soluble, high melting point waxes; and
at least one cohesion agent in an amount sufficient, at at least one temperature in the range −20 to 100 degrees Celsius, to increase the elasticity of the matrix, as assessed by increasing the breaking force of the matrix, by at least about 5% by the texture analyzer method, relative to the same matrix lacking the cohesion agent.

12. A pharmaceutical dosage form for orally administering an opioid agonist to a human, the dosage form including a matrix comprising:
a therapeutically effective amount of the opioid agonist;
at least one abuse deterrent, extended release (ADER) ingredient selected from the group consisting of hydrogenated vegetable oils, polyoxyethylene stearates, polyoxyethylene distearates, glycerol monostearate, and poorly water soluble, high melting point waxes; and
at least one cohesion agent in an amount sufficient, at at least one temperature in the range −20 to 100 degrees Celsius, to increase the stickiness of the matrix by at least about 5%, relative to the same matrix lacking the cohesion agent, as assessed by the inverted probe method.
